(19) United States
(12) Patent Application Publication
(10) Pub. No.: US 2004/0167525 A1
(43) Pub. Date: Aug. 26, 2004

(54) Anti-splay medical implant closure with multi-stepped removal counterbore

(76) Inventor: Roger P. Jackson, Prairie Village, KS (US)

Correspondence Address: John C. McMahon, PO Box 30069, Kansas City, MO, 64112, US

(21) Appl. No.: 10/783,895

(22) Filed: Feb. 20, 2004

Related U.S. Application Data
(63) Continuation-in-part of application No. 10/236,123, filed on Sep. 6, 2002, now Pat. No. 6,726,689.

(51) Int. Cl. A61B 17/58

(52) U.S. Cl. 606/73

(57) ABSTRACT

An anti-splay closure with a multi-stepped counterbore includes a cylindrical body with an external, helically extending anti-splay guide and advancement flange and a breakaway installation head. The body includes a central bore formed by a plurality of component bores diminishing in diameter in steps toward a forward end of the body to create shoulders which intersect the bores at circular edges. The circular edges form multiple locations for engagement by a reverse threaded, self-tapping fastener removal tool or easy-out. The closure is used with an open headed bone implant screw which is provided with internal, helically extending mating guide and advancement structures complementary to the flange of the body so that said body is rotatably receivable in said bone screw.

### CROSS-REFERENCE TO RELATED APPLICATION

This is a continuation-in-part of co-pending U.S. patent application, Ser. No. 10/236,123 filed Sep. 6, 2002 for HELICAL WOUND MECHANICALLY INTERLOCKING MATING GUIDE AND ADVANCEMENT STRUCTURE, which is now U.S. Pat. No. ______.

### BACKGROUND OF THE INVENTION

The present invention is directed to a closure mechanism, especially a fastener for use in closing between spaced arms in medical implants, that includes a multi-stepped internal bore for improved engagement by an “easy-out” tool for purposes of removal and an external guide and advancement structure that interlocks with mating structure on the implant arms for resisting splaying of the arms due to forces exerted during installation or later due to injury or the like.

Closure fasteners such as set screws are used in many ways in order to lock one element of a device relative to another. Such fasteners are quite important in the art of medical implants in which it is often necessary to capture one element of the implant relative to another and to then secure the two relative to one another to prevent subsequent movement therebetween. Failure to properly lock two elements of a medical implant together may result in failure of the implant and possible serious injury to the patient within which the implant is placed.

With medical implants, it is desirable to have strong and secure elements which are also very lightweight and low profile so that the overall implant impacts as little as possible upon the patient. However, strong and secure are sometimes contradictory goals compared to lightweight and low profile. Thus, size, weight and, profile must all be taken into consideration and minimized, as much as possible, consistent with effective functioning.

In order to provide sufficient strength and friction to resist movement of the various elements, once the fastener is seated, it is necessary to apply a fairly substantial torque to the fastener. While some set screws have associated driving structure that does not require a driving head and are torqued without a head, many of the fasteners currently in use in medical implants have a driving or installation head that breaks away from the remainder of the fastener at a preselected torque in order to assure that the fastener is sufficiently torqued to provide the necessary strength and locking friction. The head is also broken away in order to assure that the fastener is not over-torqued and the guide and advancement structure is not stripped. Further, the head is typically broken away in order to provide the low profile and light weight that is desired in such fasteners.

Because the driving head is typically broken away and because it is sometimes necessary to remove the fastener after implantation and setting thereof, some mechanism must be provided in order to securely engage and remove the fastener. Various structures have been provided for this purpose in prior art devices. The prior art structures have had varying degrees of success, but have typically been most effective in fasteners having a diameter that is comparatively large, such as 8 to 10 millimeters, because such larger fasteners provide greater surface and volume to allow the placement of removal structure of one kind or another on or in the fastener.

So-called “easy-outs” are self-tapping, reverse threaded extraction tools which are commonly employed to remove bolts and screws used in various mechanical devices where no other means for gripping the fastener is available. Such devices have especially been used to remove bolts of which the heads have been broken off or otherwise damaged. A bore is typically drilled into the broken-headed bolt, and the easy-out is threaded into the bore in the same direction as the direction of removal of the bolt. With proper usage and often times some degree of luck, the easy-out eventually seizes within the bore, and the easy-out and bolt, as a unit, are rotated counterclockwise to attempt to remove the bolt. The term “easy-out” is somewhat of a misnomer in that they are frequently very difficult tools to utilize. This is especially true when dealing with closures, fasteners or set screws of the size used in medical implants which often range from 5 to 10 millimeters in diameter.

It has been found that fasteners of this size with a conventional axial bore are often not removable by an easy-out, because the easy-out has too little edge or surface upon which to grip. Further, the edge that has been previously provided is often torn away by use of the easy-out, to a point where there is less and less of an edge or surface to grip with each subsequent attempt. Consequently, it is desirable to produce a closure or fastener having a head that breaks away from a base of the fastener at a preselected torque yet provides a highly gripable surface or edge in the fastener for use in conjunction with an easy-out design.

Another inherent problem in certain medical implants with closures of a conventional type is that such fasteners typically utilize threads which are referred to as V-threads or threadforms. The outer surfaces of a cross-section of V-threads form a V-shape. V-threads work reasonably well in devices where a bore is provided that completely surrounds a fastener and has a mating thread that mates with the thread of the fastener. However, many medical implants, such as open headed bone screws and open headed hooks, do not provide for a bore that will entirely encircle the closures that closes the head and locks a rod therein. In such implants, the closure spans between a pair of discontinuous threaded surfaces. When V-thread fasteners are utilized for this purpose, the forces exerted by the fastener closure during torquing are partially parallel to the axis of rotation of the closure and partially radial, extending outwardly from the closure. The radial outward forces can and frequently do spread or splay the arms of the head within which the closure is being torqued to an extent which allows the closure to slip at a torque which is less than desired.

Buttress-type threads have been utilized for the purpose of reducing the radial outward forces that are exerted by the threads. In buttress-type threads, the trailing surface of the thread normally has a cross-section with an edge that is effectively perpendicular to an axis of rotation of the closure. Sometimes such surfaces are referred to as flat, but normally the surface receiving the driving forces has a slight inclination of 5 to 10 degrees from perpendicular to the axis of rotation so that a smaller, but yet still substantial, force is exerted radially outward by the buttress thread, as compared to the V-shaped thread.

Furthermore, reverse angle threads are sometimes utilized. While such threads do not transmit an outward radial force on installation, they still provide only an interference type connection and have linear surfaces where forces are transferred, so that if an accident or the like applies strong spaying forces, the surfaces simply slide sideways and do not positively interlock or interdigitate.

Consequently, it is also desirable to provide a closure of this type including a guide and advancement structure designed to be resistant to splaying of the arms and that works in combination with other elements of the closure to allow rotation and driving for installation and rotation for removal.

### SUMMARY OF THE INVENTION

The present invention provides a fastener or closure for use particularly with an open-headed bone screw, hook or other implant. The closure has a cylindrical base and a driving or installation head that is separable from the base at a preselected torque at a breakaway region or along a breakaway line. A bore extends axially through the head and partially into the base from a trailing end thereof. The bore is multi-stepped, diminishing in diameter in steps toward a forward or leading end of the closure to form multiple bores and shoulders intersecting so as to define at least a pair of spaced circular edges. The circular edges provide multiple engagement structures for a self-tapping, reverse threaded screw removal tool, commonly known as an easy-out. The threads of the easy-out cut into the shoulders at the circular edges of the multi-stepped bore to enhance engagement of the easy-out with the closure to thereby facilitate removal of the closure from the open-headed bone screw after the installation head has been broken from a body of the closure.

In the present invention, the closure is provided with a non-threaded helical wound guide and advancement structure for securing a closure in a receiver of the bone screw. Preferably, the receiver is a rod receiving channel of an open-headed bone screw, hook, or other medical implant in which the channel has an open top and is located between two spaced apart arms forming the open head of the bone screw.

The closure body is cylindrical and has an external guide and advancement flange extending helically about the base relative to a central closure axis. The flange preferably has a compound, anti-splay type of contour which cooperates with complementary mating internal guide and advancement structures formed into the inner surfaces of the spaced apart arms forming the open head of the bone implant screw. The flange has such a compound contour as to form an inward anti-splay surface component on the closure body which faces generally inward toward the closure axis. The mating guide and advancement structures of the bone screw head have a complementary contour to the closure flange including an outward anti-splay surface component which faces, generally away from the closure axis as the closure is being installed therein.

Preferably, the inward anti-splay surface component may be formed by an enlarged region near an outer periphery of the closure flange at a crest of the flange. The outward anti-splay surface components are formed by an enlarged region near an outer periphery of the mating guide and advancement structure of the bone screw head. The complementary anti-splay surface components of the closure and head engage during insertion of the closure into the receiver between the arms by rotation thereof and then interlock and cooperate to resist splaying tendencies of the arms of the head when the closure is strongly torqued into the open head of the bone screw or when outside forces are applied due to accident, over exertion or the like.

In use, the closure and open-headed bone screw are used to anchor a spinal fixation member, such as a rod, by implanting the bone screw into a bone and clamping the rod within the head of the bone screw using the closure. In order to enhance clamping engagement of the rod, the closure body is also preferably provided with structural features which cut into the surface of the rod to thereby reduce the likelihood of translational or rotational movement of the rod relative to the bone screw. The closure is preferably provided with a centrally located set point and a peripherally located “cup point”, set ring, or V-ring on the leading end of the closure body to cut into the surface of the rod when the closure is tightly torqued into the head of the bone screw. In some embodiments, the body is also provided with a central axial point on the leading end thereof.

### OBJECTS AND ADVANTAGES OF THE INVENTION

Therefore, objects of the present invention include providing an improved closure for use with an open headed bone screw; providing such a closure having a cylindrical base and a driving or installation head that breaks away from the base at a breakaway region to provide a low or minimized profile subsequent to installation of the closure; providing such a closure having an axially extending bore that passes partially through a body of the closure from a trailing surface thereof and that has a stepdown region in the body wherein the bore passes from a region of a larger diameter to a region of a smaller diameter so as to form multiple spaced edges; providing such a closure in which the bore is multi-stepped with component bores diminishing in diameter in steps toward a forward end of the closure to provide cylindrical bores intersecting planar shoulders at respective circular edges; providing such a closure with such a multi-stepped bore to enhance secure engagement of the closure by a self-tapping, reverse threaded screw removal tool, such as an easy-out; providing such a closure in combination with an open headed bone screw implant for use in anchoring a bone fixation structural member, such as a rod; providing such a closure and implant combination in which the open headed bone screw includes a pair of spaced apart arms forming a rod receiving channel; providing such a closure and implant combination including a helical wound guide and advancement flange on a body of the closure and mating internal guide and advancement structures tapped into inner surfaces of the bone screw head which, when rotatably joined, interlock and cooperate to resist tendencies of the arms to splay when the closure is torqued tightly into clamping engagement with a rod positioned in the channel or when other forces are applied to the implant; providing such a combination including features to enhance setting engagement of the closure with a rod in the bone screw channel; providing such a combination in which a forward end of the closure is provided with a central set point and a peripheral V-ring to cut into the surface of the rod when the closure is securely torqued, to prevent translational and rotational movement of the rod relative to the bone screw; and providing such an anti-splay closure with a multi-stepped counterbore which is economical to manufacture, which is secure and efficient in use, and which is particularly well adapted for its intended purpose.

Other objects and advantages of this invention will become apparent from the following description taken in conjunction with the accompanying drawings wherein are set forth, by way of illustration and example, certain embodiments of this invention.

The drawings constitute a part of this specification, include exemplary embodiments of the present invention, and illustrate various objects and features thereof.

### BRIEF DESCRIPTION OF THE DRAWINGS

FIG. 1 is an enlarged perspective view of an anti-splay closure having an installation break off head with a multi-stepped removal counterbore in accordance with the present invention.

FIG. 2 is a side elevational view of the closure at a further enlarged scale.

FIG. 3 is a top plan view of the closure and illustrates details of the multi-stepped removal counterbore with the installation break off head in place.

FIG. 4 is a bottom plan view of the closure illustrating a set point and V-ring on a forward end of a body of the closure.

FIG. 5 is a cross sectional view of the closure taken on line 5-5 of FIG. 3 and illustrates internal details of the multi-stepped removal counterbore and helically wound guide and advancement structures.

FIG. 6 is a fragmentary side elevational view at a reduced scale of the closure in combination with an open headed bone screw.

FIG. 7 is a view similar to FIG. 6 and illustrates separation of a breakaway installation head of the closure.

FIG. 8 is a greatly enlarged cross sectional view of the closure of the present invention positioned in clamping relationship within an open headed bone screw and illustrates details of an anti-splay guide and advancement structure of and bone screw head and also illustrating an east-out tool engaging the multi-stepped removal counterbore for removing the closure body from the bone screw.

FIG. 9 is a greatly enlarged top plan view of the closure within the open headed bone screw.

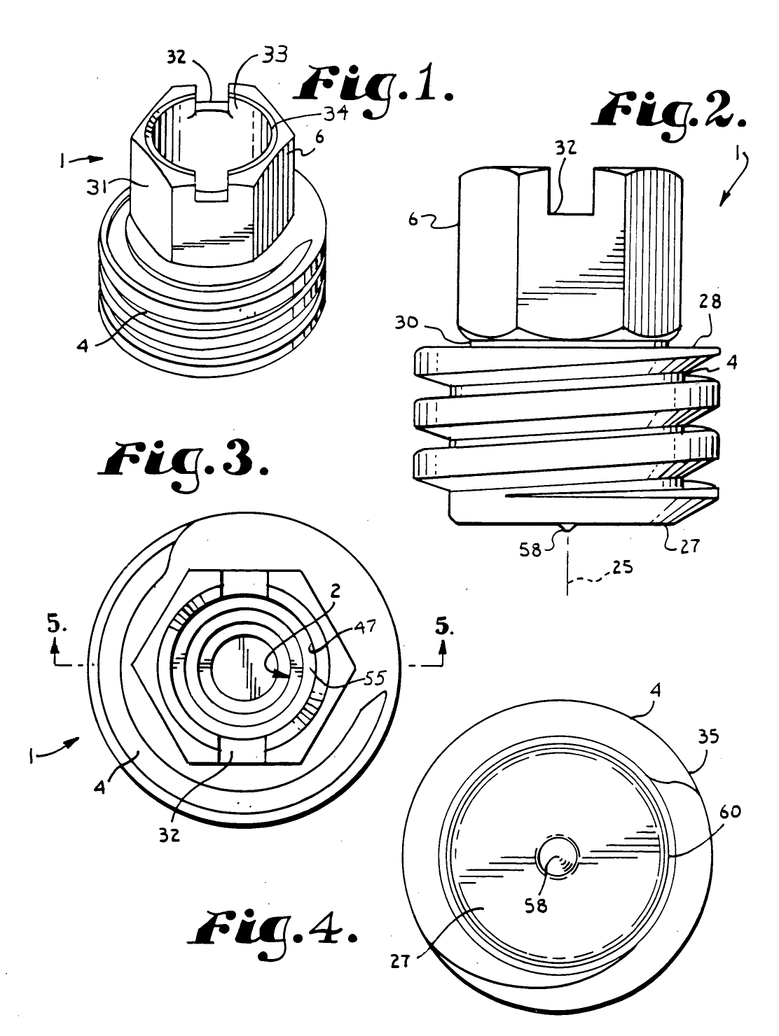

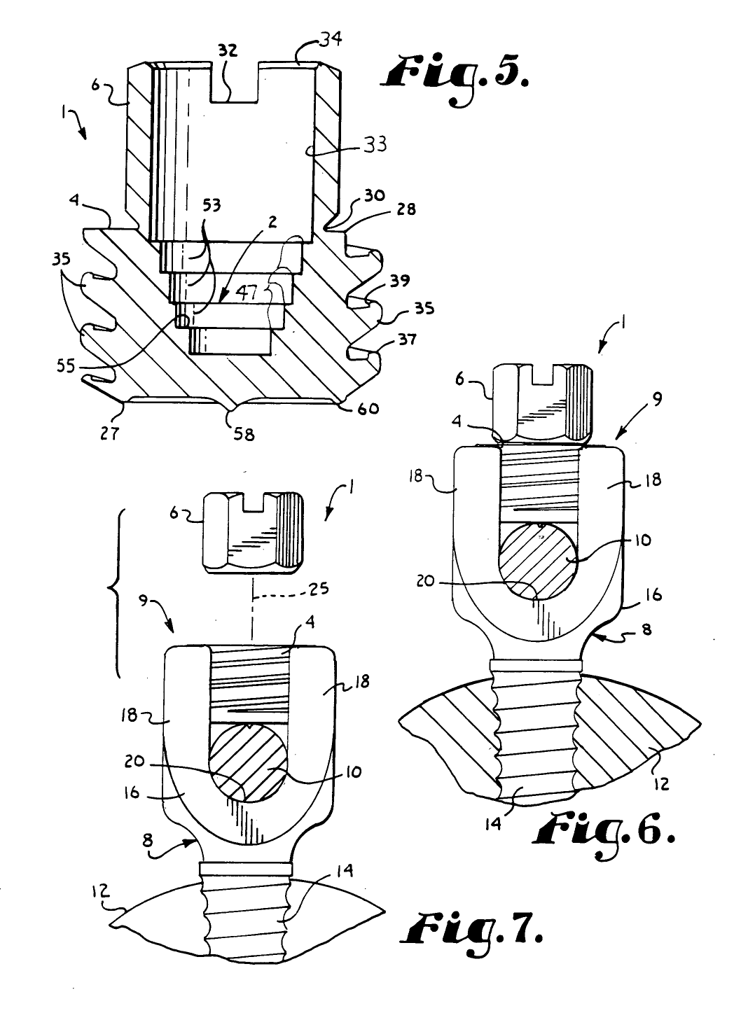

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

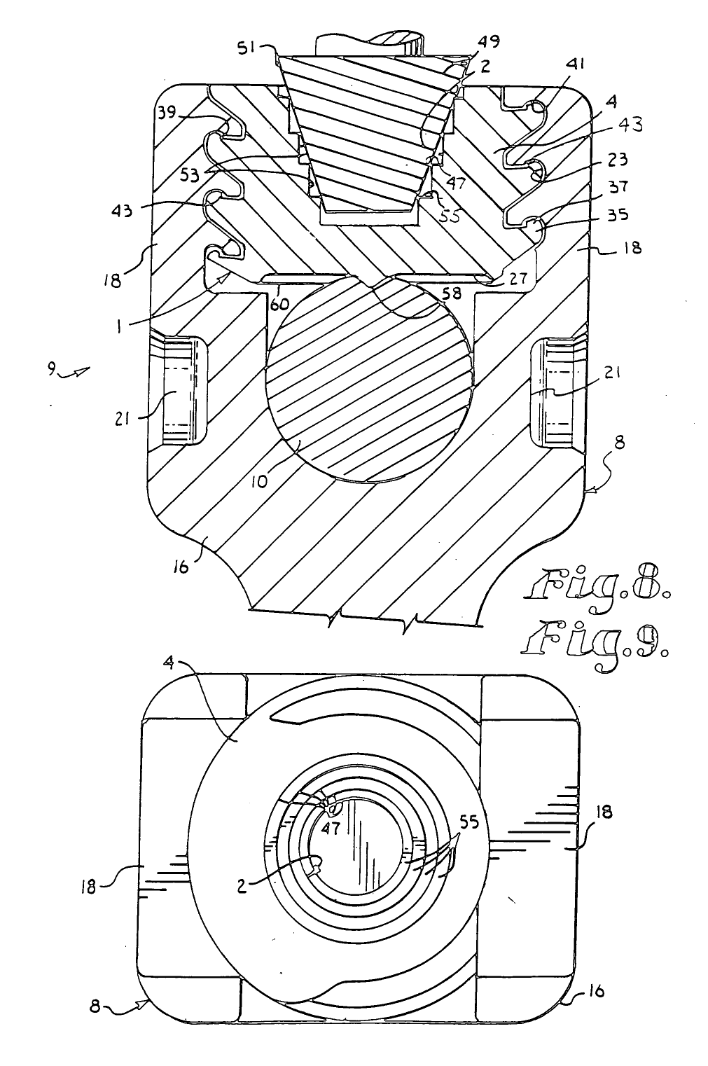

Referring to the drawings in more detail, the reference numeral 1 generally designates an anti-splay fastener or closure with a multi-stepped counterbore 2. The closure 1 generally includes a plug or body 4 and a breakaway installation head 6. The body 4 is used in cooperation with an open headed bone implant screw 8 (FIGS. 6 and 7) to form an implant anchor assembly 9 to secure or anchor a spinal fixation member or rod 10 with respect to a bone 12, such as a vertebra.

The bone screw 8 includes a threaded shank 14 for threadably implanting into the bone 12 and an open head 16 formed by a pair of spaced apart arms 18 defining a U-shaped channel 20 therebetween to receive the rod 10. Inner surfaces of the arms 18 have internal guide and advancement structures 23 (FIG. 8) tapped, or otherwise formed, therein. The head 16 has grip indentations 21 (FIG. 8) to facilitate gripping the bone screw 8 by an appropriate tool (not shown) during manipulation for implantation of the bone screw 8 into the bone 12.

The body 4 is cylindrical in external shape about a closure axis 25 (FIG. 2) and has a forward, leading, or inner end 27 and a rear, trailing, or outer end 28. The breakaway head 6 is connected to the body 4 at the rear end 28 by way of a weakened breakaway region or ring 30 formed by selectively reducing the wall thickness of the closure 1 to weaken the region. The breakaway ring 30 is thinned in such a manner that it fails at a selected relative torque between the head 6 and the body 4, as a result of torque applied to the head 6 to drive and tighten the body 4 within the bone screw 8. As illustrated, the breakaway head 6 has a hexagonal outer surface 31 to facilitate non-slip engagement by an installation tool (not shown) of a conventional socket type. The head 6 is also provided with a set of tool slots 32 for alternative or more positive non-slip engagement of the head by the installation tool. Separation of the head 6 from the body 4, as shown in FIG. 7) is desirable to control or limit torque applied by the body 4 to the rod 10 within the bone screw head 16 and to provide a low profile joint between the body 4 relative to the bone screw 8, especially where the top of the body 4, after breakoff of the head 6, is at or below the top of the arms 18.

Referring to FIG. 8, the body 4 is provided with an anti-splay guide and advancement structure or flange 35 for cooperation with the mating guide and advancement structures 23 in the open head 16 of the bone screw 8, to thereby interlock the body 4 with the head 16 to clamp the rod 10, after the body 4 is rotated into and received between the arms 18 by rotational engagement of the guide and advancement structures 35 and 23. The closure 4, after insertion in the bone screw head 16 resists splaying of the arms 18 of the head 16. The guide and advancement flange 35 extends helically about the body 4 from near the rear end 28 to near the forward end 29 thereof. The illustrated guide and advancement flange 35 has an enlarged outer bead 37 near a periphery thereof and located on a trailing surface 38 thereof, which extends along an outer periphery or crest of the guide and advancement flange 35 to form a compound contour including an inward anti-splay surface 39 which faces, or has a component or projection which faces, generally toward the body axis 25. The trailing surface 38 of the flange 35 is referenced to a forward direction of advancement of the body 4 into the bone screw 8 and is directed away from the forward end 27 of the body 4.

In a similar manner, the illustrated mating guide and advancement structures 23 on the bone screw arms 18 are enlarged near the radially outward peripheries thereof to form compound contours, on engaging surfaces 41 of the mating structures 23, including outward anti-splay surfaces 43 which face, or have components or projections which face, generally away from the body axis 25.

The inward anti-splay surfaces 39 of the body 4 engage the outward anti-splay surfaces 43 of the head 16 when the body 4 is advanced into the head 16 to resist any tendencies of the arms 18 of the head 16 to splay or be urged outward, away from the body 4, in reaction to relative torque between the body 4 and the screw head 16 or other radially acting forces that operably try to splay or separate the upper ends of the arms 18. Although the compound contours forming the anti-splay surfaces 39 and 43 are shown to be on the surfaces shown, it is conceivable that the compound contours could be formed on the leading surfaces of the body flange structure 35 and appropriate mating structure. Furthermore, the contour along the surfaces of the guide and advancement structure can be varied substantially under the invention to provide a region spaced from the closure wall and arm walls that projects axially in one direction or the other with mating structure on the opposite so that the body 4 and arms 18 are interlocked together once the body 4 is rotated into position between the arms 18. Other configurations of the interlocking flange and mating structures in accordance with the present invention are found in U.S. application for patent Ser. No. 10/236,123 which is now U.S. Pat. No. ______ and which is incorporated herein by reference.

Referring to FIGS. 5, 8, and 9, the closure 1 is provided with the multi-stepped bore or counterbore 2 to form multiple circular edges 47 for enhanced engagement by threads 49 of a reverse threaded, self-tapping closure removal tool 51, known as an easy-out.

The illustrated multi-stepped bore 2 is formed by a plurality of cylindrical component coaxial bores 53 having cylindrical surfaces which diminish in diameter in steps in a direction toward the forward end 27 of the body 4. The bores 53 create shoulders 55 which intersect successive bores 53 at the circular edges 47. The edges 47 provide locations for the self-tapping threads 49 of the easy-out 51 to cut into the shoulders 55 to more securely engage the easy-out 51 with the body 4, than if a single bore of an appropriate size and angle were employed. The easy-out 51 is threaded in a helical direction opposite that of the guide and advancement flange 35 and mating structures 23 so that threading the easy-out into the bore 2 and continued rotation in the direction of engagement by the easy-out 51 further engages the easy-out and initiates counterclockwise rotation and extraction of the body 4 from the open head 16 of the bone screw 8. The multi-stepped bore 2 increases the likelihood that the easy-out 51 will grasp the body 4 during the entire process without slippage. Once the body 4 has been loosened from the head 16, it may be rotated out of the head 16.

The body 4 also includes formations to enhance clamping and securing engagement of the body 4 with the rod 10. Referring to FIGS. 5 and 8, the illustrated body 4 includes a centrally located set point 58 and a peripherally extending V-ring or set ring 60 on the forward end 27. The set point 58 and V-ring 60, also known as a “cup point”, are provided to operably cut into the outer surface of the rod 10 when the body 4 is strongly torqued into the bone screw head 16. The point 58 and V-ring 60, when set, reduce the likelihood of rotational and translational movement between the rod 10 and the bone screw 8.

It is to be understood that while certain forms of the present invention have been illustrated and described herein, it is not to be limited to the specific forms or arrangement of parts described and shown.

What is claimed and desired to be secured by letters patent is as follows:

1. A closure for setting engagement with a structural member and comprising:
(a) a substantially cylindrical body having an outer cylindrical surface relative to a central axis;
(b) a guide and advancement flange extending helically about said outer cylindrical surface, said flange having a leading surface and a trailing surface relative to a direction of forward advancement along said axis;
(c) at least one of said leading surface and said trailing surface being compound in contour spaced from said body cylindrical surface and including an inward facing anti-splay surface component facing generally toward said body axis; and
(d) said body having a multi-stepped bore formed therein, said multi-stepped bore including a plurality of substantially cylindrical component bores sequentially diminishing in diameter in steps toward said direction of forward advancement thereby forming a plurality of cylindrical surfaces that intersect shoulders at respective circular edges sized and shaped so as to be adapted to provide a grip to an easy out tool.

2. The closure as set forth in claim 1 and including:
(a) an installation head shaped to enable non-slip engagement of said installation head by an installation tool; and
(b) said installation head being connected to said body by a breakaway region formed in such a manner that said breakaway region fails in response to a selected level of torque between said installation head and said body to enable separation of said installation head from said body.

3. The closure as set forth in claim 1 wherein:
(a) said body has a forward end relative to said direction of forward advancement; and
(b) said body having a set point formed on said forward end adapted to enhance setting engagement of said body into a surface of a structural member.

4. The closure as set forth in claim 1 wherein:
(a) said body has a forward end relative to said direction of forward advancement; and
(b) said body has a V-shaped set ring formed on said forward end adapted to enhance setting engagement of said fastener into a surface of a structural member.

5. The closure as set forth in claim 1 in combination with a bone implant screw adapted for connection to a bone fixation structural member, said bone implant screw including:
(a) a threaded shank adapted for threadably implanting into a bone;
(b) an open head formed by a pair of spaced apart arms having mutually facing channel surfaces defining a structural member receiving channel to receive a bone fixation structural member; and
(c) said mutually facing channel surfaces having respective mating guide and advancement structure formed therein which are compatible with said guide and advancement flange of said body to enable guiding and advancement of said body into said channel to thereby clamp said bone fixation structural member therein.

6. The closure and bone implant screw combination as set forth in claim 5 wherein:
(a) said mating guide and advancement structures of said bone implant screw include an outward anti-splay surface component which cooperates with said inward anti-splay surface component of said body in such a manner as to interlock and resist splaying of said arms in reaction to forces exerted on said arms.

7. The combination as set forth in claim 6 wherein:
(a) said guide and advancement flange has a relatively enlarged region near an outer periphery thereof which forms said inward anti-splay surface component;
(b) said mating guide and advancement structures are contoured in a complementary manner to said guide and advancement flange to form said outward anti-splay surface component; and
(c) said inward anti-splay surface component interlock with said outward anti-splay surface component when said body is guided and advanced into said open screw head of said bone implant screw.

8. The closure as set forth in claim 1 wherein:
(a) said cylindrical component bores of said multi-stepped bore diminish in such steps and have such cylindrical lengths that are sized and shaped so as to form said respective circular edges having locations and diameters located so as to be adapted to enhance engagement of said body by a reverse threaded, self-tapping fastener removal tool.

9. A closure for setting engagement with a structural member and comprising:
(a) a substantially cylindrical body having an outer cylindrical surface relative to a central body axis of rotation;
(b) an external guide and advancement flange extending helically about said outer cylindrical surface, said flange having a trailing surface relative to said forward advancement direction;
(c) said trailing surface being compound in contour and including an inward anti-splay surface component facing generally toward said body;
(d) said body having a multi-stepped bore formed therein, said multi-stepped bore including a plurality of substantially cylindrical component bores diminishing in diameter in steps from a rear surface of said body toward a forward direction thereof along said axis thereby forming a plurality of cylindrical surfaces that intersect shoulders at respective circular edges; and
(e) said cylindrical component bores diminishing in such steps and having such cylindrical lengths as to form said respective circular edges having locations and diameters sized and shaped so as to enhance engagement of said body by a reverse threaded, self-tapping removal tool.

10. The closure as set forth in claim 9 and including:
(a) an installation head shaped to enable non-slip engagement of said installation head by an installation tool; and
(b) said installation head being connected to said body by a breakaway region formed in such a manner that said breakaway region fails in response to a selected level of torque between said installation head and said body to enable separation of said installation head from said body.

11. The closure as set forth in claim 9 wherein:
(a) said body has a forward end relative to a forward advancement direction; and
(b) said body has a set point formed on said forward end to enhance setting engagement of said body into a surface of a structural member.

12. The closure as set forth in claim 9 wherein:
(a) said body has a forward end relative to a forward advancement direction; and
(b) said body has a V-shaped set ring formed on said forward end to enhance setting engagement of said body into a surface of a structural member.

13. The closure as set forth in claim 9 in combination with a bone implant screw adapted for connection to a bone fixation structural member, said bone implant screw including:
(a) a threaded shank adapted for threadably implanting into a bone;
(b) an open head formed by a pair of spaced apart arms having mutually facing channel surfaces defining a structural member receiving channel to receive a bone fixation structural member; and
(c) said mutually facing channel surfaces having mating internal guide and advancement structure formed therein that are compatible with rotationally mating with said flange of said body to enable advancement of said body into said channel to thereby clamp said bone fixation structural member therein.

14. The closure and bone implant screw combination as set forth in claim 13 wherein:
(a) said mating structures of said bone implant screw include an outward anti-splay surface component which cooperates with said inward anti-splay surface component of said body in such a manner as to resist a tendency of said arms to splay in reaction to forces applied to said arms.

15. The combination as set forth in claim 14 wherein:
(a) said flange has a relatively enlarged outer region near a periphery thereof which forms said inward anti-splay surface component;
(b) said mating structures are contoured in a complementary manner to said external flange to form said outward anti-splay surface component; and
(c) said inward anti-splay surface component engages said outward anti-splay surface component when said body is rotated into said open screw head of said bone implant screw.

16. A closure for setting engagement with a structural member and including a substantially cylindrical body having an outer cylindrical surface relative to a central axis and a guide and advancement flange extending helically about said outer cylindrical surface relative to said axis, said flange having a leading surface and a trailing surface relative to said forward advancement direction, and wherein:
(a) at least one of said leading surface and said trailing surface being compound in contour and including an inward anti-splay surface component facing generally toward said fastener axis; and
(b) said body having a multi-stepped bore formed therein, said multi-stepped bore including a plurality of substantially cylindrical component bores diminishing in diameter in steps toward said forward advancement direction thereby forming a plurality of cylindrical surfaces that intersect shoulders at respective circular edges.

17. The closure as set forth in claim 16 and including:
(a) an installation head sized and shaped to be adapted to enable non-slip engagement of said installation head by an installation tool; and
(b) said installation head being connected to said body by a breakaway region formed in such a manner that said breakaway region fails in response to a selected level of torque between said installation head and said body to enable separation of said installation head from said body.

18. The closure as set forth in claim 16 and including:
(a) said body having a forward end relative to said forward advancement direction;
(b) said body having a set point formed on said forward end adapted to enhance setting engagement of said body into a surface of a structural member; and
(c) said body having a V-shaped set ring formed on said forward end in surrounding relation to said set point adapted to further enhance setting engagement of said body into a surface of such a structural member.

19. The closure as set forth in claim 16 in combination with a bone implant screw adapted for connection to a bone fixation structural member, said bone implant screw including:
(a) a threaded shank adapted for threadably implanting into a bone;
(b) an open head formed by a pair of spaced apart arms having mutually facing channel surfaces defining a structural member receiving channel to receive a bone fixation structural member;
(c) said mutually facing channel surfaces having respective mating guide and advancement structures formed therein which are compatible with said guide and advancement flange of said body to enable guiding and advancement of said body into said channel to thereby be adapted to clamp said bone fixation structural member therein; and
(d) said mating guide and advancement structures of said bone implant screw including an outward anti-splay surface component which cooperates with said inward anti-splay surface component of said fastener in such a manner as to resist a tendency of said arms to splay in reaction to forces applied to said arms.

20. The combination as set forth in claim 19 wherein:
(a) said guide and advancement flange has a relatively enlarged region near an outer periphery thereof which forms said inward anti-splay surface component;
(b) said mating guide and advancement structures are contoured in a complementary manner to said guide and advancement flange to form said outward anti-splay surface component; and
(c) said inward anti-splay surface component interlocks with said outward anti-splay surface component when said body is guided and advanced into said open screw head of said bone implant screw.

21. The closure as set forth in claim 16 wherein:
(a) said cylindrical component bores of said multi-stepped bore diminish in such steps and have such cylindrical lengths as to form said respective circular edges having locations and diameters adapted to enhance engagement of said body by a reverse threaded, self-tapping fastener removal tool.
